SEQUENCE LISTING (14 sequences)

SEQ ID NO: 1 - protein, 230 aa, Mus musculus
MMPKHCLLGLLIILLSSATEIQPARVSLTPQKVRFQSRNFHNILHWQAGSSLPSNNSIYFVQYKMYGQSQWEDKVDCWGTTALFCDLTNETLDPYELYYGRVMTACAGRHSAWTRTPRFTPWWETKLDPPVVTITRVNASLRVLLRPPDLPNRNQSGKNASMETYYGLVYRVFTINNSLEKEQKAYEGTQRAVEIEGLIPHSSYCVVAEMYQPMFDRRSPRSKERCVQIP

SEQ ID NO: 2 - protein, 263 aa, Homo sapiens
MMPKHCFLGFLISFFLTGVAGTQSTHESLKPQRVQFQSRNFHNILQWQPGRALTGNSSVYFVQYKIMFSCSMKSSHQKPSGCWQHISCNFPGCRTLAKYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 3 - protein, 231 aa, Homo sapiens
MMPKHCFLGFLISFFLTGVAGTQSTHESLKPQRVQFQSRNFHNILQWQPGRALTGNSSVYFVQYKIYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 4 - protein, 130 aa, Homo sapiens
MMPKHCFLGFLISFFLTGVAGTQSTHESLKPQRVQFQSRNFHNILQWQPGRALTGNSSVYFVQYKIYGQRQWKNKEDCWGTQELSCDLTSETSDIQEPYYGRVRAASAGSYSEWSMTPRFTPWWERAKGL

SEQ ID NO: 5 - DNA, 22 bp, Artificial Sequence
  Primer
atgatgcctaagcattgccttc

SEQ ID NO: 6 - DNA, 24 bp, Artificial Sequence
  Primer
tcagaccttcaatttcaacagctc

SEQ ID NO: 7 - DNA, 28 bp, Artificial Sequence
  mIL-22BP alpha primer
cggggtaccaaggtccgatttcagtcca

SEQ ID NO: 8 - DNA, 29 bp, Artificial Sequence
  mIL-22BP alpha primer
gcggccgctcaagtcacgaccggaggatc

SEQ ID NO: 9 - DNA, 27 bp, Artificial Sequence
  mIL-22BP beta primer
cggggtacctctttgcgggtgcttctc

SEQ ID NO: 10 - DNA, 29 bp, Artificial Sequence
  mIL-22BP beta primer
gcggccgctcacatttcagccactacgca

SEQ ID NO: 11 - DNA, 309 bp, Mus musculus
aaggtccgatttcagtccagaaatttccacaatattttgcactggcaagctgggagctctctccccagcaacaacagcatctactttgtgcagtacaagatgtatggacagagccaatgggaagataaagttgactgctgggggaccacggcgctcttctgtgacctgaccaatgaaaccttagacccatacgagctgtattacgggagggtgatgacggcctgtgctggacgccactctgcctggaccaggacaccccgcttcactccatggtgggaaacaaaactagatcctccggtcgtgacttga

SEQ ID NO: 12 - protein, 102 aa, Mus musculus
KVRFQSRNFHNILHWQAGSSLPSNNSIYFVQYKMYGQSQWEDKVDCWGTTALFCDLTNETLDPYELYYGRVMTACAGRHSAWTRTPRFTPWWETKLDPPVVT

SEQ ID NO: 13 - DNA, 216 bp, Mus musculus
tctttgcgggtgcttctccgtcctccagagttgccaaatagaaaccaaagtggaaaaaatgcatccatggaaacttactacggcttagtatacagagttttcacaatcaacaattcactagagaaggagcaaaaagcctatgaaggaactcagagagctgttgaaattgaaggtctgatgcctcattccagctactgcgtagtggctgaaatgtga

SEQ ID NO: 14 - protein, 71 aa, Mus musculus
SLRVLLRPPELPNRNQSGKNASMETYYGLVYRVFTINNSLEKEQKAYEGTQRAVEIEGLMPHSSYCVVAEM

(19) United States
(12) Patent Application Publication
(10) Pub. No.: US 2009/0148440 A1
(43) Pub. Date: Jun. 11, 2009

(54) ANTIBODIES AGAINST INTERLEUKIN-22 BINDING PROTEIN AND ITS USES FOR THE TREATMENT OF METABOLIC DISORDERS

(75) Inventors: Yu Liang Huang, Shanghai (CN); Xu Wen Chen, Shanghai (CN); Zhi Hua Huang, Shanghai (CN)

Correspondence Address: EAGLE IP LIMITED, 22/F., KWAI HUNG HOLDINGS CENTRE, 89 KING'S ROAD, NORTH POINT, HK

(73) Assignee: DHY & CO., LTD., Tortola, Virgin Islands (GB)

(21) Appl. No.: 12/096,180

(22) PCT Filed: Dec. 21, 2006
(86) PCT No.: PCT/US2006/062450

§ 371 Date: Jun. 4, 2008

Related U.S. Application Data
(60) Provisional application No. 60/752,397, filed on Dec. 22, 2005.

(51) Int. Cl. A61K 39/395; C07K 16/18

(52) U.S. Cl. 424/133.1; 530/388.23; 530/389.2; 530/387.3; 424/158.1; 424/145.1; 424/142.1

(57) ABSTRACT

The present invention relates to antibodies and antigen-binding fragments that bind to interleukin-22 binding protein, in particular, human interleukin-22 binding protein (IL-22 BP), and are involved in regulating interleukin-22-associated biological responses. The invention also relates to methods of using the antibodies and antigen-binding fragments to treat disorders associated with interleukin-22. The antibodies disclosed herein are useful in diagnosing, preventing, or treating metabolic disorders including obesity, diabetes, hyperlipidemia and hyperinsulinemia etc.

### FIELD OF THE INVENTION

The invention is related generally to the cytokine interleukin-22. In particular, the present invention relates to antibodies and antigen-binding fragments that bind to and neutralize the interleukin-22 binding protein.

### BACKGROUND OF THE INVENTION

Interleukin-22 (IL-22) or IL-TIF (Interleukin 10-related T cell-derived inducible factor) is a cytokine structurally related to IL-10 and originally identified as the product of a gene induced by IL-9 in murine T lymphocytes (Dumoutier, L. et al. 2000. Proc. Natl. Acad. Sci. USA 97:10144). In vitro, the expression of IL-22 was found in T helper cells upon stimulation with IL-9, anti-CD3 Abs, or Concanavilin A (Con A) and in IL-9-stimulated mast cells. In vivo, IL-22 production is demonstrated in spleen cells upon anti-CD3 and Lipopolysaccaride (LPS) administration. The biological role IL-22 was proposed to be involved in inflammatory processes and a number of metabolic disorders, such as obesity, diabetes, hyperlipidemia and hyperinsulinemia.

IL-22 activates its specific receptor complexes on its target cells. The IL-22 receptor complex consists of two chains. One chain, IL-22RA was specific to IL-22 binding. The other chain, IL-10Rβ is shared with IL-10. IL-10Rβ, also called CRF2-4, is required for IL-10 signaling. IL-22RA was also described as CRF2-9, an orphan receptor called ZCYTOR11 in patent databases and proposed to be renamed IL-22R by Xie et. al. (2000. J. Biol. Chem., 275:31335). Both chains of IL-22 receptor belong to the class II cytokine receptor family.

A genetically distinct soluble receptor for IL-22 in human has recently been identified. This soluble receptor is 40 kDa in size and 210 amino acids (aa) in length. It was named as soluble IL-22 R/CRF2-10, also called IL-22 receptor-α 2 or IL-22 binding protein (IL-22BP). Two alternate splice forms have been reported in addition to the standard 210 aa form, One splice form is truncated with 131 aa in length. The second splice form is a 242 aa mature molecule. The long form was found in placenta and may regulate both IL-22 and IL-20 activity. The standard form, 210 aa of IL-22 binding protein was shown to be an antagonist for IL-22 activity. IL-22BP is able to bind to IL-22 and neutralizes the bioactivities of IL-22 demonstrated in BaF3 cells expressing IL-22 receptor subunit (Xu et al. PNAS, 2001, vol 98:9511-9516), STAT activation in IL-22-responsive human lung carcinoma A549 cells, induction of the suppressors of cytokine signaling-3 (SOCS-3) expression in HepG2 cells (Kotenko et al. Journal of Immunology, 2001 vol 166:7096-7103).

### SUMMARY OF THE INVENTION

The present invention provides antibodies, particularly an isolated humanized antibody, or isolated human antibody or a biologically active portion thereof that specifically binds to and blocks the bioactivity of human (IL-22BP).

In one aspect, an antibody of the invention binds to IL-22BP and modulates the interaction between human IL-22 and IL-22BP. Such modulation blocks the interaction of IL-22 with IL-22BP, thus to increase the levels of free IL-22 molecules capable of binding the IL-22 receptor complexes led to the enhanced biological responses of target tissue or cells to IL-22.

In one embodiment, the antibody of the invention is polyclonal and specifically inhibits the interaction between IL-22 and IL-22BP. In another embodiment, the antibody of the invention is monoclonal and specifically inhibits the interaction between IL-22 and IL-22BP.

In the most preferred embodiment, the antibody of the invention is an isolated antibody or biologically active portion thereof that is capable of binding and neutralizing the bioactivities of IL-22 binding protein and its variants as shown in Seq 2, 3 and 4.

In another aspect, the invention is a method of treating metabolic disorders comprising administering a therapeutically effective dose of an anti-IL-22 binding protein antibody.

Yet in another aspect of the present invention relates to a pharmaceutical composition comprising an isolated and purified antibody. It also relates to a pharmaceutical composition consisting essentially of an isolated and purified antibody.

### BRIEF DESCRIPTION OF FIGURES

FIG. 1 is a full length murine IL-22BP sequence.

FIG. 2 is a murine IL-22BP alpha sequence.

FIG. 3 is a murine IL-22BP beta sequence.

FIG. 4 is a sequence alignment of full-length mIL-22BP, mIL-22BP alpha and mIL-22BP beta.

FIG. 5 is Protein A purified anti IL-22BP antibodies.

FIG. 6 is a graph showing reduced serum triglyceride (TG) levels in response to IL-22BP in vivo.

### DETAILED DESCRIPTION OF PREFERRED EMBODIMENTS

As used herein and in the claims, the term “humanized antibody” is defined as being a human antibody composed of over 50% human peptide sequence, preferably over 70%, and most preferably over 90% human peptide sequence, and which causes minimal antigenicity when injected into a human at therapeutically effective doses. The preferred embodiment of the present invention is a human antibody and a peptibody with a specific peptide binding domain and a human Fc region. However, this invention may comprise any proteins that are capable of binding IL-22BP while also blocking the interaction of IL-22BP with IL-22. Such proteins may be, and are not limited to, a polyclonal antibody, monoclonal antibody, chimeric antibody, humanized antibody, human antibody, antigen-binding fragment, or any peptide with a human Fc fragment etc.

A randomly generated peptides with the Fc domain is known as a “Peptibody”, See U.S. Pat. No. 6,660,843, issued Dec. 9, 2003, to Feige et al. (incorporated by reference in its entirety). They include on or more peptides linked to the N-terminus, C-terminus, amino acid side chains, or to more than one of these sites. Peptibody technology enables design of therapeutic agents that incorporate peptides that target one or more ligands or receptors, tumor-homing peptides, membrane-transporting peptides, and the like. Peptibody technology has proven useful in design of a number of such molecules, including linear and disulfide-constrained peptides, “tandem peptide multimers” (i.e., more than one peptide on a single chain of an Fc domain). See, for example, U.S. Pat. No. 6,660,843; U.S. Pat. App. No. 2003/0195156, published Oct. 15, 2003 (corresponding to WO 02/092620, published Nov. 21, 2002); U.S. Pat. App. No. 2003/0176352, published Sep. 18, 2003 (corresponding to WO 03/031589, published Apr. 17, 2003); U.S. Ser. No. 09/422,838, filed Oct. 22, 19999 (corresponding to WO 00/24770, published May 4, 2000); U.S. Pat. App. No. 2003/0229023, published Dec. 11, 2003; WO 03/057134, published Jul. 17, 2003; U.S. patent application Ser. No. 10/666,480, filed Sep. 18, 2003 (corresponding to WO 04/026329, published Apr. 1, 2004), each of which is hereby incorporated by reference in its entirety.

The following examples teach the production of the antibody of the invention and demonstrate the effectiveness of such antibody. All references cited are incorporated in their entirety herein.

### EXAMPLES

### 1. Murine IL-22 Binding Protein (IL-22BP) Gene Cloning

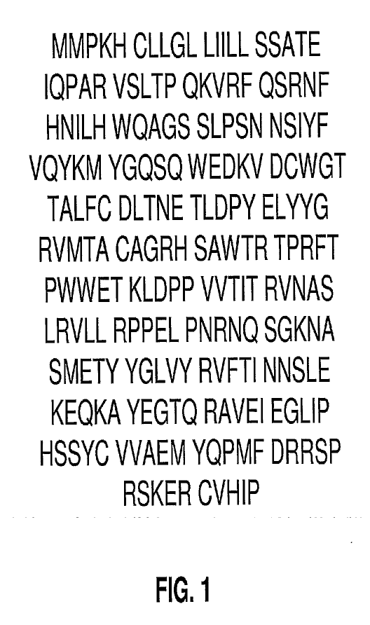

The cloning of murine IL-22 binding protein gene (FIG. 1, nucleotide sequence Seq 1) used a similar protocol as described by Weiss, B. et al (Genes Immun. 5:330-336, 2004, GenBank Accession number: AJ555484). Briefly, total RNA from mouse spleen was extracted using a Qiagen RNeasy isolation kit (Qiagen GmbH). Full-length murine IL-22BP cDNA was cloned using the Qiagen OneStep RT-PCR kit. PCR amplification used gene-specific primers 5′-atg atg cct aag cat tgc ctt c-3′ (Seq 5), and 5′-tca gac ctt caa ttt caa cag ctc-3′ (Seq 6). PCR-products were cloned into PCR4 vector (Invitrogen) vector and confirmed by sequence analysis.

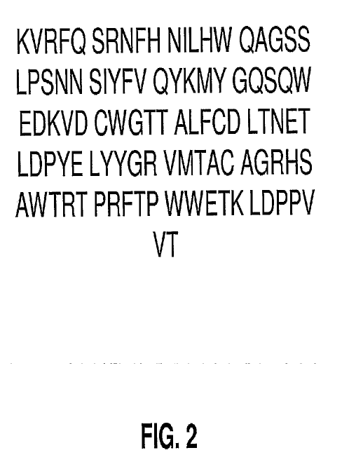

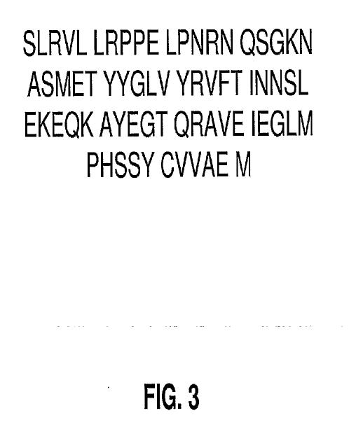

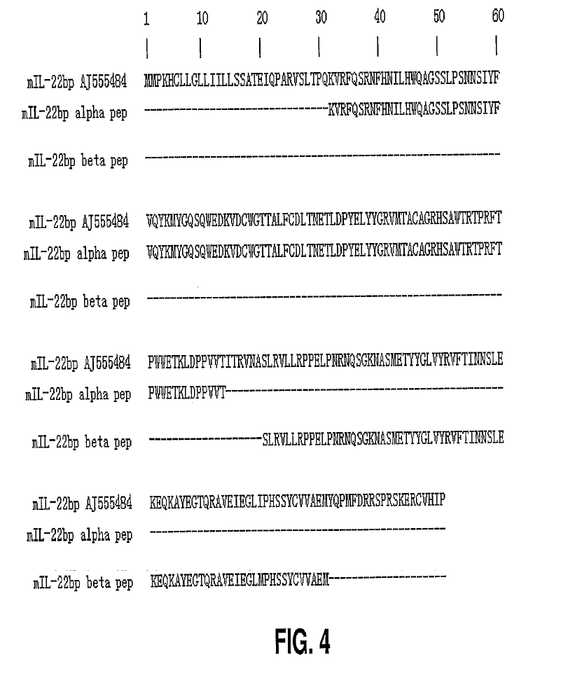

Two subclones of mIL-22BP cDNA were made using the full-length cDNA as template. The first clone, mIL-22BP alpha, contained amino acid 32 to 133 without the putative signal sequence (FIG. 2). The second clone, mIL-22BP beta contained amino acid 140 to 210 (FIG. 3). The sequence comparison of full-length murine IL-22BP and the subclones are shown in FIG. 4. The mIL-22BP alpha sequence was cloned using PCR primers 5′-cgg ggt acc aag gtc cga ttt cag tcc a-3′ (Seq 7) and 5′-gcg gcc gct caa gtc acg acc gga gga tc-3′ (Seq 8). The mIL-22BP beta sequence was cloned using PCR primers 5′-cgg ggt acc tct ttg cgg gtg ctt ctc-3′ (Seq 9) and 5′-gcg gcc gct cac att tca gcc act acg ca-3′ (Seq 10). The amplified DNA fragments were cloned to the pMD18-T (Takara) and plasmids were prepared. Plasmids containing mIL-22BP alpha and mIL-22BP beta were digested with Not I and Kpn I and cloned into expression vector pET32a (Novagen). The sequences of mIL-22BP alpha and mIL-22BP beta were confirmed by DNA sequence analysis as shown in Seq 11 and 13, respectively.

### 2. Recombinant mIL-22BP Alpha and mIL-22BP Beta Protein Expression

The expression of the recombinant mIL-22BP alpha and mIL-22BP beta used a similar method as described by Wei Chi-Chen et al. (Genes and Immunity vol: 4:p 204, 2003). Briefly, E. Coli strain BL21(+) (Stratagene) was used as the expression host. The host cells were cultured in Luria-Bertani (LB) medium with ampicillin (100 ug/mL). Expression of the protein was induced with 1 mM isopropyl-b-D-thiogalactoside. The cell pellet was disrupted with a homogenizer, and the mIL-22BP alpha and mIL-22BP beta inclusion bodies were obtained by centrifugation. Inclusion bodies were washed with TriszHCl 50 mM, NaCl 100 mM, EDTA 1 mM, DTT 1 mM, and sodium deoxycholate 0.5% (wt/vol), pH 8. Inclusion bodies were solubilized overnight at 4° C. in 100 mM NaH₂PO₄, 10 mM Tris-HCl, and 8M Urea, pH 8.0. The solution was centrifuged for 30 mins 100,000×g and the supernatant collected. The recombinant mIL-22BP proteins were purified using Ni-NTA agarose chromatography using Ni-NTA spin kit (Qiagen GmbH, Germany). The purified mIL-22BP proteins were treated with enterokinase (Invitrogen) to remove the thioredoxin fusion protein. The purity of the mIL-22BP alpha (Seq 12) and mIL-22BP beta (seq 14) proteins was estimated >90% based on SDS-PAGE and Coomassie blue staining analysis.

### 3. Preparation of anti-mIL-22BP Antibodies

Rabbits were used to produce polyclonal antibodies against mIL-22BP. The recombinant mIL-22BP alpha and mIL-22BP beta protein were mixed together at (1:1 by weight) ratio to immunize rabbits. The immunizing solution contained a mixture of 1.5 mg mIL-22BP alpha and 1.5 mg mIL-22BP beta proteins in 2.0 mL PBS plus 2.0 mL Complete Freund's Adjuvant (CFA, Sigma) as described in Current Protocol in Immunology, Edited by Coligan et al 1994. Each rabbit received 2.0 mL immunizing solution by subcutaneous injection at 4 sites on the back of the rabbit. After the first immunization, rabbits were again subcutaneously injected on week 3 and 6 with the same amount of proteins plus Incomplete Freund's Adjuvant (IFA, Sigma). Serum samples were collected in week 6 and the antibody titers were determined by ELISA (Current Protocol in Immunology, Edited by Coligan et al 1994). The antibodies titers determined in both rabbits were higher than 1:1×10⁶ against both mIL-22BP alpha and mIL-22BP beta.

TABLE 1
Characterization of rabbit anti-mouse IL-22BP antibodies.

| | | Antibody Titer | |
|---|---|---|---|
| Rabbit | Serum sample | mIL-22BP alpha | mIL-22BP beta |
| 1 | Pre-immunization | 2,000 | 2,000 |
| | Post-immunization | >1,024,000 | 1,024,000 |
| 2 | Pre-immunization | 1,000 | 1,000 |
| | Post-immunization | >1,024,000 | >>1,024,000 |

### 4. Protein A Purified Anti IL-22BP Antibodies

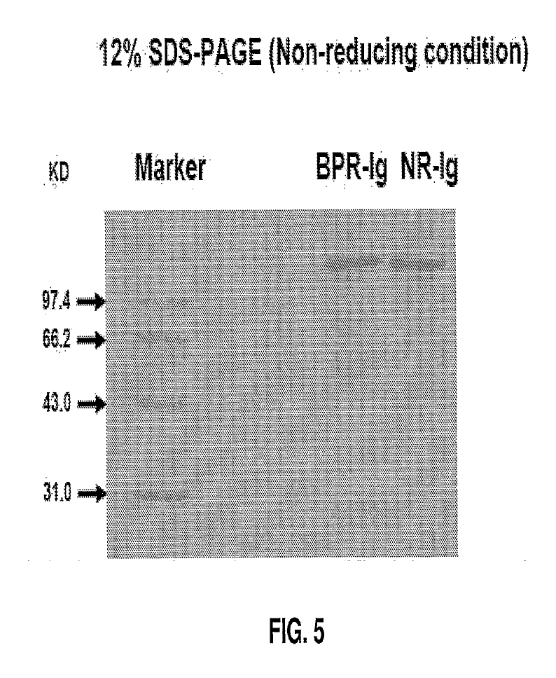

Antibody Serum Immunoglobulin (IgG) from a normal rabbit and an immunized rabbit were purified using Protein A Sepharose column (Current Protocol in Immunology, Edited by Coligan et al 1994). The purified IgG was dissolved in PBS and kept at 4° C. FIG. 5 shows a SDS-PAGE gel containing IgG purified from normal rabbit without immunization (NR-Ig) and IgG purified from mIL-22BP immunized rabbit (BPR-Ig). The purity of protein A column chromatography was estimated to be higher than 95%.

### 5. Anti-IL-22BP Antibodies Block the Bioactivity of IL-22 In Vivo

Female C57BL/6 mice, age 16 weeks, body weight 19 to 24 grams were treated with single injection (subcutaneously) of protein A-purified rabbit polyclonal antibodies (IgG) against the murine IL-22 binding protein at dose of 0.1 mg or 1.0 mg per animal in 0.2 mL of PBS. The control mice received purified immunoglobulin (IgG) isolated from rabbits that were not immunized with antigens. The control group received the same dose of IgC, that is, 0.1 mg or 1.0 mg per animal in 0.2 mL of PBS. Serum were harvested from the treated mice on the 7^{th} day after injection and stored at −20° C. The serum levels of triglyceride (TG) were determined using an automated blood chemistry analyzer (Synchron Lxi 725, Beckman Coulter Inc. USA].

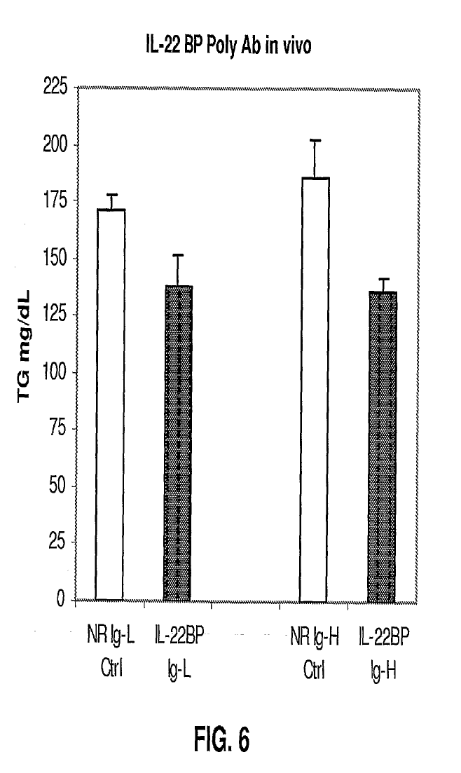

Results: The serum levels of TG are shown in Table 2. Mice treated with purified control IgG had normal levels of serum TG Mice treated with purified IgG from rabbits immunized with recombinant mIL-22BP alpha plus mIL-22BP beta resulted in significantly reduced serum levels of TG (p=0.049 and p=0.018). The results show that blocking IL-22 binding protein in vivo using neutralizing antibodies can significantly reduce the serum levels of TG (FIG. 6).

TABLE 2
Effects of blocking IL-22 binding protein in vivo by neutralizing
antibodies.

| | TG mg/dL | | | |
|---|---|---|---|---|
| Group | (mean, D7) | n | D7 SEM | p value |
| NR Ig-L Ctrl (A) | 171.57 | 8 | 6.93 | |
| IL-22BP Ig-L (B) | 138.38 | 8 | 13.15 | 0.049 (A vs B) |
| NR Ig-H Ctrl (C) | 185.84 | 8 | 17.08 | |
| IL-22BP Ig-H (D) | 135.73 | 8 | 5.9 | 0.018 (C vs D) |

NR: normal rabbit;
Ig-L: low dose at 0.1 mg per mice;
Ig-H: high dose at 1.0 mg per mice;
Ctrl: control mice;
SEM; standard error of the mean;
p-values were calculated using student t-test.

The preferred embodiments of the present invention are thus fully described. Although the description referred to particular embodiments, it will be clear to one skilled in the art that the present invention may be practiced with variation of these specific details. Hence, this invention should not be construed as limited to the embodiments set forth herein.

For example, a person skilled in the art will appreciate that the present invention may be employed in the binding and neutralizing of bioactivities of other IL-22 binding protein and its variants as shown in Seq 2, 3 and 4.

1. An isolated and purified antibody (polyclonal or monoclonal) that specifically binds to human interleukin-22 binding protein and its derivatives thereof.

2. The isolated and purified antibody of claim 1, wherein said antibody specifically inhibits the binding of IL-22 binding protein to IL-22.

3. The isolated and purified antibody of claim 2, wherein said antibody is used for the diagnosis, prevention or treatment of a metabolic disorder selected from the group consisting of: obesity, diabetes, hyperlipidemia and hyperinsulinemia.

4. The isolated and purified antibody of claim 3, wherein said antibody is polyclonal antibody, monoclonal antibody, chimeric antibody, antigen-binding fragment, any peptide with a Fc fragment, or any derivatives thereof.

5. The isolated and purified antibody of claim 4 wherein said antibody is humanized.

6. The isolated and purified antibody of claim 4 wherein said antibody is fully human antibody generated in mice by transgenic or gene targeting technologies.

7. The isolated and purified antibody of claim 5 wherein said antibody is composed of over 50% human peptide sequence.

8. A pharmaceutical composition comprising an isolated and purified antibody, wherein said antibody specifically binds to human interleukin-22 binding protein and its derivatives thereof.

9. The pharmaceutical composition of claim 8 wherein said antibody specifically inhibits the binding of IL-22 binding protein to IL-22.

10. The pharmaceutical composition of claim 9 wherein said antibody is used for diagnosis, prevention or treatment of a metabolic disorder selected from the group consisting of: obesity, diabetes, hyperlipidemia and hyperinsulinemia.

11. The pharmaceutical composition of claim 10 wherein said antibody is polyclonal antibody, monoclonal antibody, chimeric antibody, antigen-binding fragment, any peptide with a Fc fragment, or any derivatives thereof.

12. The pharmaceutical composition of claim 11 wherein said antibody is humanized.

13. The pharmaceutical composition of claim 11 wherein said antibody is fully human antibody generated I mice by transgenic or gene targeting technologies.

14. The pharmaceutical composition of claim 12 wherein said antibody is composed of over 50% human peptide sequence.

15. A method of making a pharmaceutical composition comprising an isolated and purified antibody and a pharmaceutically acceptable carrier wherein said antibody specifically binds to human interleukin-22 binding protein and its derivatives thereof.

16. A method of treatment of a human comprising administering a pharmaceutically effective dose of an isolated and purified antibody wherein said antibody specifically binds to human interleukin-22 binding protein and its derivatives thereof.

17. The method of treatment of a human of claim 16 wherein said antibody is used for treatment of a metabolic disorder selected from the group consisting of: obesity, diabetes, hyperlipidemia and hyperinsulinemia.
